(19) United States
(12) Patent Application Publication
(10) Pub. No.: US 2021/0236296 A1
(43) Pub. Date: Aug. 5, 2021

(54) SPINAL DISC REPLACEMENTS AND METHODS OF MAKING THEREOF

(71) Applicant: TDBT IP Inc., New York, NY (US)

(72) Inventors: Scott Douglas CORNEZ, New York, NY (US); Jerson MEZQUITA, Bronx, NY (US); Nathaniel BACHRACH, Clifton, NJ (US)

(73) Assignee: TDBT IP Inc., New York, NY (US)

(21) Appl. No.: 17/213,600

(22) Filed: Mar. 26, 2021

Related U.S. Application Data
(63) Continuation of application No. PCT/US2019/053274, filed on Sep. 26, 2019.
(60) Provisional application No. 62/737,915, filed on Sep. 27, 2018.

(51) Int. Cl. A61F 2/44; A61F 2/30

(52) U.S. Cl. CPC A61F 2/442; A61F 2/3094; A61F 2/30771; A61F 2002/30772; A61F 2002/30985; A61F 2002/302; A61F 2002/30062

(57) ABSTRACT

A spine disc replacement composition, biocompatible support structure, and methods of fabricating the spine disc replacement and biocompatible support structure are provided. The spine disc replacement composition includes the biocompatible support structure that includes one or more of an annular ring, a first plate, or second plate of a biocompatible material and a tissue-engineered construct that includes a bio ink, where the annular ring includes an inner surface, an outer surface, a first planar surface, and a second planar surface, and the biocompatible material is present in an amount of about 1% to about 100% by weight of the biocompatible support structure.

### CROSS-REFERENCE TO RELATED PATENT APPLICATIONS

This application claims the benefit of and priority to U.S. Provisional Application No. 62/737,915 filed Sep. 27, 2018, and PCT International Application No. PCT/US2019/053274, filed Sep. 26, 2019, which are incorporated herein by reference in their entireties.

### TECHNICAL FIELD

The present technology relates generally to spine disc replacements and methods of preparing the same. More particularly, the present technology relates to spine disc replacements having a biocompatible support structure and tissue-engineered construct and methods of fabricating via 3D printing or injection molding.

### BACKGROUND

Spinal discs can become damaged due to herniation, bulging, degeneration, or trauma, and they do not heal on their own. Current surgical options for fixing damage to spinal discs include synthetic discs, cages, or fusion of vertebrae. Fully synthetic discs face issues with staying in place and integration into the body, possibly leading to further surgeries. Fusion of vertebrae inhibits a patient's range of motion and does not restore quality of life. The present technology is directed to overcoming these and other deficiencies. In addition, the spine disc replacements of the present technology maintain the structural integrity of tissue-engineered constructs, such as tissue-engineered intervertebral discs.

### SUMMARY

In one aspect, the present technology provides a spine disc replacement composition that includes a biocompatible support that includes one or more of an annular ring, a first plate, or second plate of a biocompatible material structure and a tissue-engineered construct that includes a bio ink, where the annular ring includes an inner surface, an outer surface, a first planar surface, and a second planar surface, and the biocompatible material is present in an amount of about 1% to about 100% by weight of the biocompatible support structure.

In another aspect, the present technology provides a biocompatible support structure that includes one or more of an annular ring, a first plate, or a second plate of a biocompatible material; where the annular ring includes an inner surface wall, an outer surface wall, a first planar surface, and a second planar surface; and the biocompatible material is present in an amount of about 1% to about 100% by weight of the biocompatible support structure.

In a related aspect, the present technology provides a method of fabricating the biocompatible support structure as described herein in any embodiment, the method includes depositing a biocompatible material to a substrate; optionally crosslinking the deposited biocompatible material; and optionally repeating the depositing and optional crosslinking steps to obtain the biocompatible support structure; where the biocompatible support structure includes one or more of an annular ring, a first plate, or a second plate of a biocompatible material, the annular ring includes an inner surface wall, an outer surface wall, a first planar surface, and a second planar surface, and the biocompatible material is present in an amount of about 1% to about 100% by weight of the biocompatible support structure.

In yet another related aspect, the present technology provides a method of fabricating the spine disc replacement that includes fabricating a biocompatible support structure as described herein in any embodiment comprising: depositing a biocompatible material to a substrate; optionally crosslinking the deposited biocompatible material; and optionally repeating the depositing and optional crosslinking steps to obtain the biocompatible support structure; fabricating a tissue-engineered construct as described herein in any embodiment comprising: depositing a bio ink as described herein in any embodiment in or around the biocompatible support structure; crosslinking the bio ink, and optionally repeating the depositing and crosslinking steps, to form the tissue-engineered construct; and curing the spine disc replacement composition; where the biocompatible support structure includes one or more of an annular ring, a first plate, or a second plate of a biocompatible material, the annular ring includes an inner surface wall, an outer surface wall, a first planar surface, and a second planar surface, and the biocompatible material is present in an amount of about 1% to about 100% by weight of the biocompatible support structure.

### BRIEF DESCRIPTION OF THE DRAWINGS

FIG. 1A is a perspective view of a biocompatible support structure having an annular ring according to an embodiment.

FIG. 1B is a top view, cross-sectional view, and side view of a biocompatible support structure having an annular ring according to an embodiment.

FIG. 1C is a side view of a biocompatible support structure having an annular ring according to an embodiment.

FIG. 2 is a top view and side views of a biocompatible support structure having an annular ring according to an embodiment.

FIG. 3 is a top view and side views of a biocompatible support structure having a first (or second) plate according to an embodiment.

FIG. 4 is a top view and side views of a biocompatible support structure having a first (or second) plate according to an embodiment.

FIG. 5 is a top view, perspective view, and side view of a biocompatible support structure having a first (or second) plate and one or more appendage elements according to an embodiment.

FIG. 6 is a top view, side view, and perspective view of a biocompatible support structure having a first plate and second plate having appendage elements (i.e., struts) connecting a planar surface of the first plate and second plate according to an embodiment.

FIG. 7 is a top view, side views, and perspective view of a spine disc replacement according to an embodiment.

FIG. 8 is a top view, side views, and perspective view of a tissue-engineered construct according to an embodiment.

FIG. 9 is a flow chart depicting an exemplary method for fabricating a biocompatible support structure and tissue-engineered construct of the spine disc replacement.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “intervertebral disc” refers to discs separating the spinal vertebrae from one another and acts as natural shock absorbers by cushioning impacts and absorbing stress and strain transmitted to the spinal column. Intervertebral disc tissues are primarily composed of three regions, the end plates, the annulus fibrosus and the nucleus pulposus. The annulus fibrosus is a tough collagen-fiber composite that has an outer rim of type I collagen fibers surrounding a less dense fibrocartilage and a transitional zone. These collagen fibers are organized as cylindrical layers. In each layer the fibers are parallel to one another; however, the fiber orientation between layers varies between 30 and 60 degrees. This organization provides support during torsional, bending, and compressive stresses on the spine. The end plates, which are found at the upper and lower surfaces of the disc, work in conjunction with the annulus fibrosus to contain the gel-like matrix of the nucleus pulposus within the intervertebral disc. The nucleus pulposus is made up of a soft matrix of proteoglycans and randomly oriented type II collagen fibers in water. The proteoglycan and water content are greatest at the center of the disc and decrease toward the disc periphery. Tissues that effectively mimic these structures can be produced according to the methods described herein. These collagen fibers are organized as cylindrical layers.

The term “biocompatible material” refers to a material derived from a natural or synthetic source having the ability to perform its desired function in the body of a subject without eliciting any undesirable local or systemic effects. As used herein, biocompatible materials may also refer to materials that are biodegradable, bioabsorbable, bioresorbable, or a combination of two or more thereof under physiological conditions. The term “biodegradable” refers to a material that can be broken down into basic substances through normal environmental processes and/or by the action of living things, such as, microorganisms. The term “bioabsorbable” refers to a material capable of being absorbed into living tissue. The term “bioresorbable” refers to a material that upon placement within the human body starts to dissolve (resorbed) and slowly replaced by advancing tissue. As used herein, in any embodiment, the terms “biodegradable,” “bioabsorbable,” and “bioresorbable” are used interchangeably.

As used herein, a “subject” or a “patient” is a mammal as described herein. The term “subject” and “patient” can be used interchangeably. As used herein, the term mammal includes, but is not limited to, a cat, dog, rodent, or primate. For example, in any embodiment herein, the mammal is a human.

The term “hydrogel” or “gel” refers to a substance formed when an organic polymer (natural or synthetic) is set or solidified to create a three-dimensional open-lattice structure that entraps molecules of water or other solution to form a gel. The solidification may occur, e.g., by aggregation, coagulation, hydrophobic interactions, or crosslinking. Hydrogels may be cellular (i.e., containing cells) or acellular (i.e., without cells). Hydrogels containing cells may rapidly solidify to keep the cells evenly suspended within a mold (or around or within another solidified gel) until the gel solidifies. Hydrogels may also be biocompatible, e.g., not toxic to cells suspended in the hydrogel.

As used herein, the term “collagen” refers to the main protein of connective tissue that has a high tensile strength and that has been found in most multicellular organisms. Collagen is a major fibrous protein, and it is also the non-fibrillar protein in basement membranes. It contains an abundance of glycine, proline, hydroxyproline, and hydroxylysine. Collagen is found throughout the body and is of at least 12 types (type I-XII).

### Spine Disc Replacement of the Present Technology

In one aspect, the present technology provides a spine disc replacement that includes a biocompatible support structure that includes one or more of an annular ring, a first plate, or second plate of a biocompatible material and a tissue-engineered construct that includes a bio ink, where the annular ring includes an inner surface, an outer surface, a first planar surface, and a second planar surface, and the biocompatible material is present in an amount of about 1% to about 100% by weight of the biocompatible support structure.

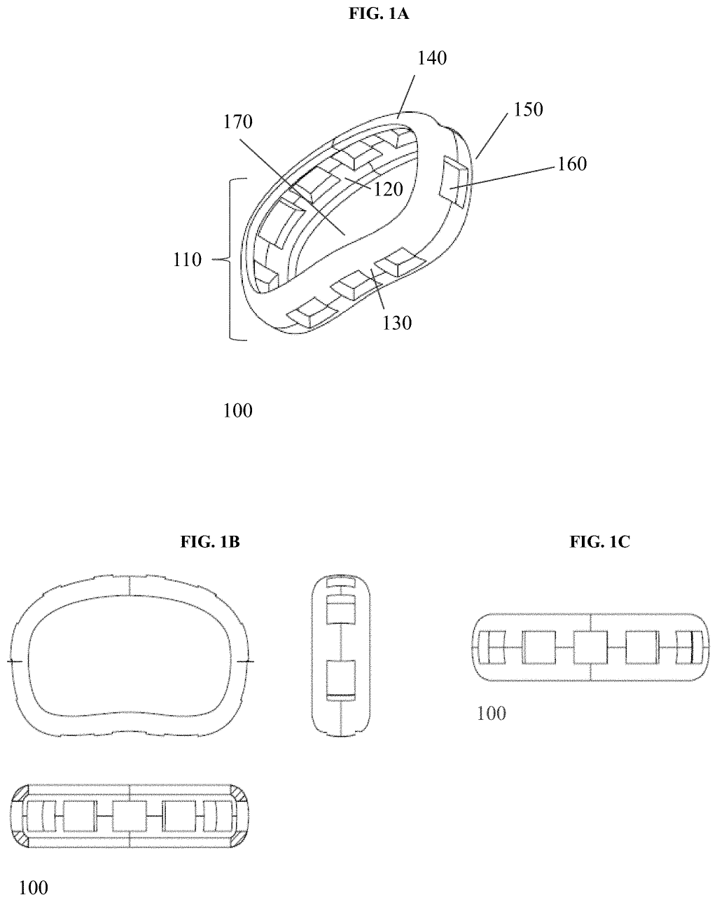

FIG. 1A provides a perspective view of a biocompatible support structure 100 according to an embodiment that includes an annular ring 110, where the annular ring has an inner surface wall 120, an outer surface wall 130, a first planar surface 140, and a second planar surface 150. The biocompatible support structure 100 further illustrates one or more apertures 160 extending-there through from the outer surface wall 130 to the inner surface wall 120, in fluid communication with external physiological environment of the annular ring 110 to the interior cavity 170 of the annular ring 110. FIGS. 1B and 1C illustrate a top view, cross-sectional view, and side views of a biocompatible support structure 100.

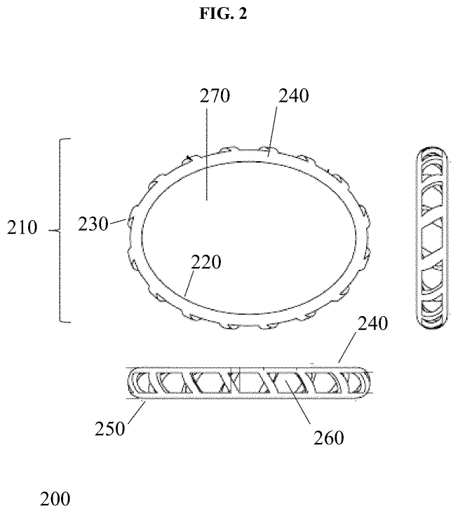

FIG. 2 provides a top view and side views of a biocompatible structure 200 according to an embodiment that includes an annular ring 210, where the annular ring has an inner surface wall 220, an outer surface wall 230, a first planar surface 240, and a second planar surface 250. The biocompatible support structure 200 further illustrates one or more apertures 260 extending-there through from the outer surface wall 230 to the inner surface wall 220, in fluid communication with the external physiological environment of the annular ring 210 to the interior cavity 270 of the annular ring.

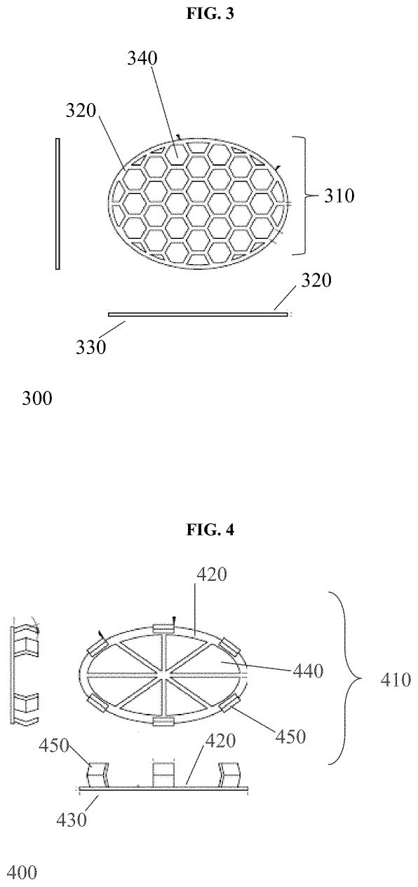

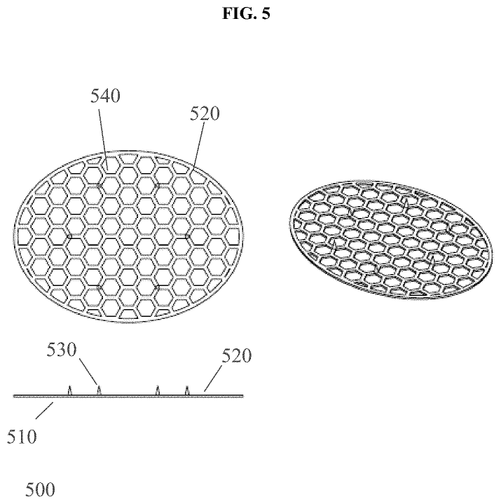

FIG. 3 provides a top view and side views of a biocompatible support structure 300 according to an embodiment that includes a first (or second) plate 310, where the plate has a first planar surface 320 and a second planar surface 330. The first (or second) plate 310 includes one or more apertures 340 extending-there through from the first planar surface to the second planar surface. FIG. 4 provides a top view and side views of a biocompatible support structure 400 according to an embodiment that includes a first (or second) plate 410, where the plate has a first planar surface 420 and a second planar surface 430. The first (or second) plate 410 includes one or more apertures 440 extending-there through from the first planar surface to the second planar surface. The first (or second) plate further includes one or more appendage elements 450 connected to and extending distally from the first planar surface 420. FIG. 5 provides a top view and side view of a biocompatible support structure 500 according to an embodiment that includes a first (or second) plate, where the plate has a first planar surface 510 and a second planar surface 520. The first (or second) plate includes one or more apertures 540 extending-there through from the first planar surface to the second planar surface. The first (or second) plate further includes one or more appendage elements (i.e., spikes) 530 connected to and extending distally from the second planar surface 520.

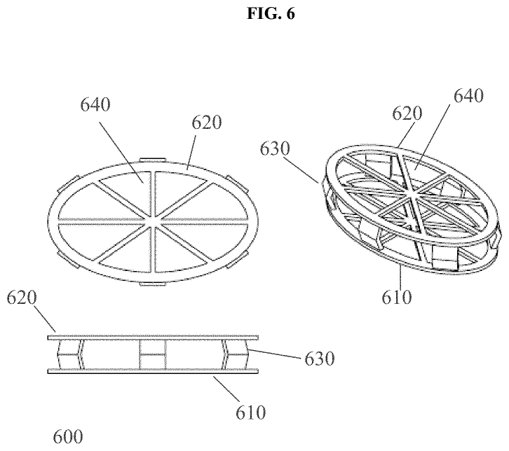

FIG. 6 provides a top view, perspective view, and side view of a biocompatible support structure 600 according to an embodiment that includes a first plate 610 and second plate 620. The first (or second) plate includes one or more apertures 640 extending-there through. The first and/or second plate further include one or more appendage elements (i.e., struts) 630 connecting the a planar surface of the first plate 610 to the second plate 620.

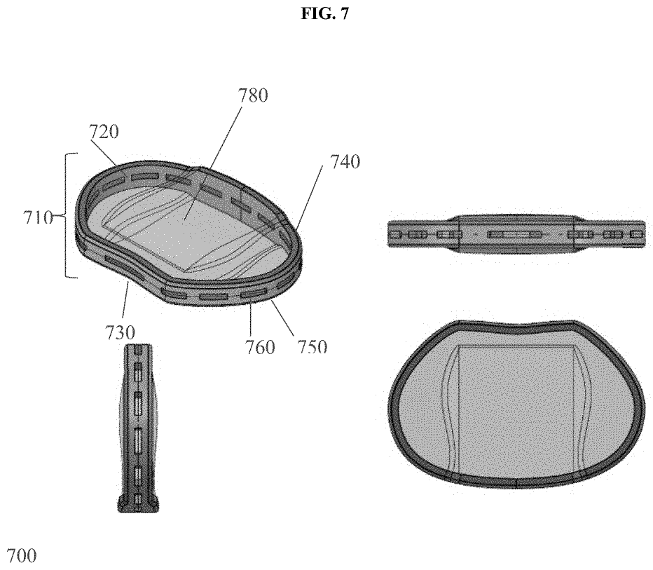

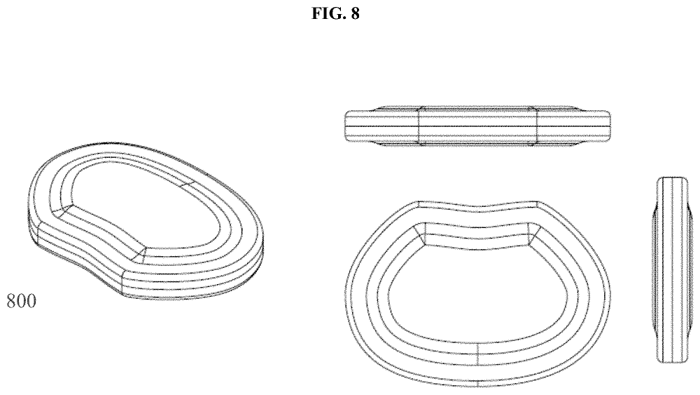

FIG. 7 provides a top view, perspective view, and side views of a spine disc replacement 700 according to an embodiment that includes an annular ring 710. The annular ring 710 includes an inner wall 720, outer wall 730, first planar surface 740, and second planar surface 750. The annular ring further includes one or more apertures 760 extending-there through to the tissue-engineered construct 780, allowing fluid communication between the external physiological environment and the tissue-engineered construct.

FIG. 8 provides a top view, perspective view, and side views of a tissue-engineered construct 800 according to an embodiment having an annular (IVD-like) shape.

### Biocompatible Support Structure

In one aspect, the present technology provides a biocompatible support structure that includes one or more of an annular ring, a first plate, or a second plate of a biocompatible material. As used herein, “annular ring” refers to a ring- or hoop-like shape that may be configured to have a circular, ovular, or elliptical shapes. For example, in any embodiment herein, the ring- or hoop-like shape may include a cavity (i.e., opening) extending-there through the interior of the annular ring to allow the flow of materials (e.g., nutrients, biological waste material, cellular and tissue materials, or the like or combinations thereof). In any embodiment herein, the annular ring may have a circumferential shape of an intervertebral disc (IVD) of a subject. For example, in any embodiment herein, one or more portions of the annular ring may have a profile that may be flat, concave, convex, or a combination thereof. FIG. 1A-1C provides an embodiment of biocompatible support 100 that includes an annular ring 110, where one or more portions of the annular ring 110 having a concave and convex profile.

The annular ring may include an inner surface wall, an outer surface wall, a first planar surface, and a second planar surface. As used herein, “inner surface wall” refers to a surface of the annular ring along any point facing the interior cavity of the annular ring. As used herein, “outer surface wall” refers to a surface of the annular ring along any point facing the exterior, or opposite side, of the interior of the annular ring. As used herein, “planar surface” refers to surfaces that are generally recognized as flat or capable of being laid flat. For example, in any embodiment herein, a generally planar surface may include undulations, deviations, or textural elements (e.g., nodules, spikes, divots, or the like or combinations thereof). For example, in an embodiment, FIG. 1A provides a perspective view of a biocompatible support structure 100 that includes an annular ring 110, where the annular ring has an inner surface wall 120, an outer surface wall 130, a first planar surface 140, and a second planar surface 150. FIG. 2 provides a top view and side views of a biocompatible structure 200 according to an embodiment that includes an annular ring 210, where the annular ring has an inner surface wall 220, an outer surface wall 230, a first planar surface 240, and a second planar surface 250.

Without being bound by theory, it is believed that the annular ring bolsters the axial stiffness of the tissue-engineered construct due to the circumferential compression of the tissue-engineered construct by the annular ring. The circumferential compression applied by the annular ring prevents or reduces the degree of bulging of the tissue-engineered construct in response to compressive force, improving the axial stiffness of the tissue-engineered construct. In any embodiment herein, the annular ring of the biocompatible support structure increases the axial stiffness of the tissue-engineered construct by 5 times its stiffness to about 10,000 times its stiffness. For example, in any embodiment herein, the axial stiffness may be increased by about 5 times, about 10 times, about 20 times, about 30 times, about 40 times, about 50 times, about 60 times, about 70 times, about 80 times, about 90 times, about 100 times, about 200 times, about 300 times, about 400 times, about 500 times, about 1000 times, about 5000 times, about 10,000 times, or any range including and/or in between any two of the preceding values.

In any embodiment herein, the first planar surface and second planar surface of the annular ring are opposite each other, and the annular ring has a medial thickness orthogonal to the planar surfaces. For example, in any embodiment herein, the annular ring has a medial thickness of about 100 μm to about 6000 μm. Suitable medial thicknesses include, but are not limited to, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1250 μm, about 1500 μm, about 1750 μm, about 2000 μm, about 2250 μm, about 2500 μm, about 2750 μm, about 3000 μm, about 3250 μm, about 3500 μm, about 3750 μm, about 4000 μm, about 4250 μm, about 4500 μm, about 4750 μm, about 5000 μm, about 5250 μm, about 5500 μm, about 5750 μm, about 6000 μm, or ranges including and/or in between in two of the preceding values. For example, in any embodiment herein, the medial thickness may be about 100 μm to about 6000 μm, about 1000 μm to about 6000 μm, about 3000 μm to about 6000 μm, about 4000 μm to about 6000 μm, or ranges including and/or in between any two of the preceding values.

In any embodiment herein, the annular ring may have a lateral thickness orthogonal to the inner and outer surface walls. For example, in any embodiment herein, the annular ring has a lateral thickness of about 100 μm to about 2000 μm. Suitable lateral thicknesses include, but are not limited to, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1100 μm, about 1200 μm, about 1300 μm, about 1400 μm, about 1500 μm, about 1600 μm, about 1700 μm, about 1800 μm, about 1900 μm, about 2000 μm, or ranges including and/or in between in two of the preceding values. For example, in any embodiment herein, the medial thickness may be about 100 μm to about 2000 μm, about 300 μm to about 1500 μm, about 500 μm to about 1000 μm, about 700 μm to about 1000 μm, about 500 μm to about 750 μm, or ranges including and/or in between any two of the preceding values. In any embodiment herein the inner surface or outer surface of the peripheral wall may be configured to be flat, concave, convex, or a combination thereof.

In any embodiment herein, the annular ring may include one or more apertures extending-there through from the outer surface wall to the inner surface wall of the annular ring allowing the flow of materials (e.g., nutrients, biological waste material, cellular and tissue materials, or the like or combinations thereof). The one or more apertures may have an average size in a range from about 10 μm to about 10,000 μm. FIG. 1A shows an embodiment of a biocompatible support structure 100 having one or more apertures 160 extending-there through from the outer surface wall 130 to the inner surface wall 120, in fluid communication with the external physiological environment of the annular ring 110 to the interior cavity 170 of the annular ring 110. In any embodiment herein, the average size of the one or more apertures may include, but is not limited to, about 10 μm, about 20 μm, about 30 μm, about 40 μm, about 50 μm, about 60 μm, about 70 μm, about 80 μm, about 90 μm, about 100, about 200 μm, about 300 μm, about 400 μm, about 500 μm, about 600 μm, about 700 μm, about 800 μm, about 900 μm, about 1000 μm, about 1500 μm, about 2000 μm, about 2500 μm, about 3000 μm, about 3500 μm, about 4000 μm, about 4500 μm, about 5000 μm, about 5500 μm, about 6000 μm, about 6500 μm, about 7000 μm, about 7500 μm, about 8000 μm, about 8500 μm, about 9000 μm, about 9500 μm, about 10,000 μm, or any range including and/or in between any two the preceding values. Suitable ranges include from about 10 μm to about 10,000 μm, about 500 μm to about 7500 μm, about 1000 μm to about 7000 μm, about 2000 μm to about 5000 μm, about 3500 μm to about 6500 μm, or ranges including and/or in between any two of the preceding values. The one or more apertures may have any shape; for example, the shape of the one or more apertures may be circular, ovular, elliptical, polygonal, or the like or combinations thereof. In any embodiment herein, the first planar surface or the second planar surface may include one or more appendage elements connected to and extending distally from the first planar surface and/or second planar surface of the annular ring. For example, in any embodiment herein, the appendage elements may include, but are not limited to, spikes, nodules, nodes, struts, ridges, or the like or combinations thereof.

In any embodiment herein, the annular ring may withstand a hoop stress of about 1 MPa to about 100 MPa. For example, in any embodiment herein, the annular ring may withstand a hoop stress of about 1 MPa, about 2 MPa, about 3 MPa, about 4 MPa, about 5 MPa, about 6 MPa, about 7 MPa, about 8 MPa, about 9 MPa, about 10 MPa, about 15 MPa, about 20 MPa, about 30 MPa, about 40 MPa, about 50 MPa, about 60 MPa, about 70 MPa, about 80 MPa, about 90 MPa, about 100 MPa, or any range including and/or in between any two of the preceding values.

In any embodiment herein, the first plate and the second plate may have a generally planar configuration, including two generally planar surfaces opposite each other and a thickness generally orthogonal to the planar surfaces. For example, in any embodiment herein, the planar surfaces of the first plate and second plate may include undulations, deviations, or textural elements (e.g., nodules, spikes, divots, or the like or combinations thereof). For example, in an embodiment, FIG. 3 provides a top view and side views of a biocompatible support structure 300 that includes a first (or second) plate 310, where the plate has a first planar surface 320 and a second planar surface 330. In any embodiment herein, the first plate and the second may have a thickness of about 100 μm to about 1200 μm. For example, in any embodiment herein, the first plate and the second plate may have a thickness of about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, or any range including and/or in between any of the preceding values. Suitable thicknesses include, but are not limited to, from about 100 μm to about 600 μm, about 200 μm to about 600 μm, about 400 μm to about 600 μm, about 100 μm to about 300 μm, or ranges including and/or in between any of the preceding values.

The first plate and second plate may include one or more apertures extending-there through allowing the flow of materials (e.g., nutrients, biological waste material, cellular and tissue materials, or the like or combinations thereof). The one or more apertures may have an average size in a range from about 10 μm to about 10,000 μm as described herein in any embodiment. For example, suitable ranges include from about 10 μm to about 10,000 μm, about 500 μm to about 7500 μm, about 1000 μm to about 7000 μm, about 3500 μm to about 6500 μm, or ranges including and/or in between any two of the preceding values. The one or more apertures may have any shape; for example, the shape of the one or more apertures may be circular, ovular, elliptical, polygonal, or the like or combinations thereof. For example, FIGS. 3 and 4 provide top views of a first (or second) plate 310, 410 that includes one or more apertures 340, 440 extending-there through from the first planar surface 320, 420 to the second planar surface 330, 430. In any embodiment herein, a planar surface of the first plate or second plate may include one or more appendage elements connected to and extending distally from the planar surface of the first plate or second plate. For example, in any embodiment herein, the appendage elements may include, but are not limited to, spikes, nodules, nodes, struts, ridges, or the like or combinations thereof. In any embodiment herein, the one or more appendage elements may connect the first plate to the second plate. FIG. 4 shows a biocompatible support structure that includes a first (or second) plate 410 that includes one or more appendage elements 450 connected to and extending distally from the first planar surface 420.

In any embodiment herein, the first plate, second plate, or combination thereof may increase axial load capacity of the tissue-engineered construct by about 1% to about 10,000%. For example, in any embodiment herein, the axial load capacity of the tissue-engineered construct may be increased by about 1% to about 10,000%, about 1% to about 1,000%, about 1% to about 100%, about 1% to about 10%, or any range including and/or in between any two of the preceding values. Suitable increases in axial load capacity may include, but are not limited to, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 4,000%, about 5,000%, about 6,000%, about 7,000%, about 8,000%, about 9,000%, about 10,000%, or any range including and/or in between any two of the preceding values. Without being bound by theory, it is believed that the first plate and/or second plate distribute the axial load of the tissue-engineered construct in response to a compressive force.

In any embodiment herein, the biocompatible support structure may include any combination of the annular ring, the first plate, or the second plate. For example, the biocompatible support structure may include only the annular ring as described herein in any embodiment. The biocompatible support, in any embodiment herein, may include only the first plate. In any embodiment herein, the biocompatible support structure may include the annular ring and the first plate, where a planar surface of the first plate is in contact with the first or second planar surface of the annular ring. For example, the first plate may be connected to the first or second planar surface of the annular ring such that the first plate fully or partially covers one-side of the interior cavity of the annular ring. In any embodiment herein, the biocompatible support structure may include the annular ring, the first plate, and the second plate. For example, the a planar surface of the first plate may be in contact with the first planar surface of the annular ring, and a planar surface of the second plate may be in contact with the second planar surface of the annular ring, such that the first and second plates fully or partially cover the interior cavity of the annular ring on both sides. In any embodiment herein, the first plate and/or second plate may be incorporated as a planar surface contiguous with a border of the first and/or second planar surface of the annular ring. In any embodiment herein, the biocompatible support structure may include the first plate and the second plate where a planar surface of the first plate and the second plate is in contact with the tissue-engineered construct.

The biocompatible support structure may include one or more linking elements. For example, in any embodiment herein, the one or more linking elements may include rods, struts, beams, or the like or combinations thereof. In any embodiment herein, the linking elements may connect a cross-sectional area of the annular ring from one point of contact of the inner surface wall to another point of contact of the inner surface wall. In any embodiment herein, the linking elements may connect a planar surface of the first plate to a planar surface of the second plate. The one or more linking elements may be configured to form a truss-like structure within the annular ring or between the first plate and the second plate. For example, in any embodiment herein, the truss-like structure may include a Warren truss, an octet truss, a Pratt truss, a bowstring truss, a King post truss, a lenticular truss, a Town's lattice truss, a Vierendeel truss, or the like or a combination of two or more thereof. In any embodiment herein, the truss-like structure may be 2-dimensional in forming one or more inner surfaces or one or more outer surfaces. In any embodiment herein, the truss-like structure may be 3-dimensional in design to provide a continuum of support. In addition the one or more linking elements may be combined such that the planar surfaces of the first plate or second plate may be connected, or multiple concentric annular rings may be employed.

The biocompatible support structure may include a biocompatible material. For example, in any embodiment herein, the biocompatible material may include, but is not limited to, polysaccharides, biocompatible polymers, rubber, silicon, biocompatible metals (e.g., steel, cobalt-chromium alloys, titanium, titanium alloys magnesium, magnesium alloys, zinc, zinc alloys, iron alloys, or the like or combinations thereof), biocompatible ceramics, polyethylene glycol, polypropylene glycol, poly amino acids, natural and biopolymers (e.g., glycosaminoglycans, cellulose, chitosan, chitin, dextrans, gelatin, collagen, lignins, polyamino acids, glycoproteins, eslastin, laminins), or combinations of two or more thereof. In any embodiment, the biocompatible material may include biocompatible polymers; for example, in any embodiment herein, the biocompatible polymers may include polylactides (PLA), polyglycolides acid (PGA), poly (lactide-co-glycolide) (PLGA), polydioxanone (PDO), polycaprolactones, and combinations thereof. In any embodiment herein, the biocompatible polymer may have a melting point between about 50° C. to about 290° C. For example, in any embodiment herein, the biocompatible polymer may have a melting point of about 50° C., about 55° C., about 60° C., about 65° C., about 70° C., about 75° C., about 80° C., about 85° C., about 90° C., about 95° C., about 100° C., about 110° C., about 120° C., about 130° C., about 140° C., about 150° C., about 160° C., about 170° C., about 180° C., about 190° C., about 200° C., about 210° C., about 220° C., about 230° C., about 240° C., about 250° C., about 260° C., about 270° C., about 280° C., about 290° C., or any range including and/or in between any two of the preceding values.

In any embodiment herein, the biocompatible material may further include one or more additives. For example, the one or more additives may include, but are not limited to crosslinking agents.

The biocompatible material may be biodegradable, bioabsorbable, bioresorbable, or a combination thereof. For example, in any embodiment herein, the biocompatible material may degrade, absorb, or resorb under physiological conditions at a rate of from about 1 month to about 7 years. Suitable rates of degradation, absorption, or resorption may include, but are not limited to, about 1 month, about 2 months about 3 months, about 4 months, about 5 months, about 6 months about 7 months, about 8 months, about 9 months, about 10 months, about 11 months, about 1 year, about 1.5 years, about 2 years, about 2.5 years, about 3 years, about 3.5 years, about 4 years, about 4.5 years, about 5 years, about 5.5 years, about 6 years, about 6.5 years, about 7 years, or ranges including and/or in between any two of the preceding values. For example, in any embodiment herein, the rate of degradation, absorption, or resorption may include about 1 month to about 7 years, about 1 month to about 5 years, about 1 month to about 3 years, about 1 month to about 1 year, about 1 month to about 6 months, about 1 month to about 3 months. Suitable biodegradable, bioabsorbable, and/or bioresorbable materials include, but are not limited to, include, but are not limited to, PLA, PGA, PLGA, PDO, polycaprolactones, bioresorbable metal alloys, and combinations thereof.

The biocompatible material may have a density of about 1500 mg/ml to about 2500 mg/ml. For example, in any embodiment herein, the biocompatible material may have a density of about 1500 mg/ml, about 1600 mg/ml, about 1700 mg/ml, about 1800 mg/ml, about 1900 mg/ml, about 2000 mg/ml, about 2100 mg/ml, about 2200 mg/ml, about 2300 mg/ml, about 2400 mg/ml, about 2500 mg/ml, or any range including and/or between any two of the preceding values. In any embodiment herein, the biocompatible material may have a density of about 1500 mg/ml to about 2500 mg/ml, about 1750 mg/ml to about 2200 mg/ml, about 2000 mg/ml to about 2300 mg/ml, or any range including and/or in between any two of the preceding values.

The biocompatible material may be present in the biocompatible support structure in an amount of about 1% by weight to 100% by weight of the biocompatible support structure. For example, in any embodiment herein, the amount of biocompatible material present by weight in the biocompatible support structure may be about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or any range including and/or in between any two of the preceding values. Suitable amounts of the bio compatible material in the biocompatible support include, but are not limited to, about 5% to about 100%, 10% to about 100%, about 50% to about 100%, about 5% to about 20%, about 90% to about 100%, or any range including and/or in between any two of the preceding values.

The biocompatible material may be present in the biocompatible support structure in an amount of about 1% by weight to 100% by weight of the biocompatible support structure. For example, in any embodiment herein, the amount of biocompatible material present by weight in the biocompatible support structure may be about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or any range including and/or in between any two of the preceding values. Suitable amounts of the bio compatible material in the biocompatible support include, but are not limited to, about 5% to about 100%, 10% to about 100%, about 50% to about 100%, about 5% to about 20%, about 90% to about 100%, or any range including and/or in between any two of the preceding values.

The biocompatible support structure has mechanical properties allowing it to bolster mechanical properties of the tissue-engineered construct. In any embodiment herein, the biocompatible support structure may have one or more of a flexural modulus of about 0.2 GPa to about 100 GPa, a modulus of elasticity of about 0.02 GPa to about 100 GPa, or a tensile strength of about 1 MPa to about 1000 MPa.

In any embodiment herein, the biocompatible support structure may have a flexural modulus of about 0.2 GPa to about 100 GPa. For example, in any embodiment herein, the biocompatible support structure may have a flexural modulus of about 0.2 GPa, about 0.3 GPa, about 0.4 GPa, about 0.5 GPa, about 0.6 GPa, about 0.7 GPa, about 0.8 GPa, about 0.9 GPa, about 1 GPa, about 2 GPa, about 3 GPa, about 4 GPa, about 5 GPa, about 6 GPa, about 7 GPa, about 8 GPa, about 9 GPa, about 10 GPa, about 11 GPa, about 12 GPa, about 13 GPa, about 14 GPa, about 15 GPa, about 16 GPa, about 17 GPa, about 18 GPa, about 19 GPa, about 20 GPa, about 25 GPa, about 30 GPa, about 35 GPa, about 40 GPa, about 45 GPa, about 50 GPa, about 55 GPa, about 60 GPa, about 65 GPa, about 70 GPa, about 75 GPa, about 80 GPa, about 85 GPa, about 90 GPa, about 95 GPa, about 100 GPa, or any range including and/or in between any two of the preceding values. Suitable flexural modulus values may include, but are not limited to about 0.2 GPa to about 100 GPa, about 0.2 GPa to about 50 GPa, about 0.2 GPa to about 25 GPa, about 0.2 GPa to about 15 GPa, about 1 GPa to about 15 GPa, about 5 GPa to about 15 GPa, or ranges including and/or in between any two of these values.

In any embodiment herein, the biocompatible support structure may have a modulus of elasticity of about 0.02 GPa to about 100 GPa. For example, in any embodiment herein, the biocompatible support structure may have a modulus of elasticity of about 0.02 GPa, 0.04 GPa, about 0.06 GPa, about 0.08 GPa, about 0.1 GPa, about 0.2 GPa, about 0.3 GPa, about 0.4 GPa, about 0.5 GPa, about 0.6 GPa, about 0.7 GPa, about 0.8 GPa, about 0.9 GPa, about 1 GPa, about 2 GPa, about 3 GPa, about 4 GPa, about 5 GPa, about 6 GPa, about 7 GPa, about 8 GPa, about 9 GPa, about 10 GPa, about 11 GPa, about 12 GPa, about 13 GPa, about 14 GPa, about 15 GPa, about 16 GPa, about 17 GPa, about 18 GPa, about 19 GPa, about 20 GPa, about 25 GPa, about 30 GPa, about 35 GPa, about 40 GPa, about 45 GPa, about 50 GPa, about 55 GPa, about 60 GPa, about 65 GPa, about 70 GPa, about 75 GPa, about 80 GPa, about 85 GPa, about 90 GPa, about 95 GPa, about 100 GPa, or any range including and/or in between any two of the preceding values. Suitable modulus of elasticity values may include, but are not limited to about 0.2 GPa to about 100 GPa, about 0.2 GPa to about 50 GPa, about 0.2 GPa to about 25 GPa, about 0.2 GPa to about 15 GPa, about 1 GPa to about 15 GPa, about 5 GPa to about 15 GPa, or ranges including and/or in between any two of these values.

In any embodiment herein, the biocompatible support structure may have a tensile strength of about 1 MPa to about 1000 MPa. For example, in any embodiment herein, the biocompatible support structure may have a tensile strength of about 1 MPa, about 2 MPa, about 3 MPa, about 4 MPa, about 5 MPa, about 6 MPa, about 7 MPa, about 8 MPa, about 9 MPa, about 10 MPa, about 20 MPa, about 25 MPa, about 30 MPa, about 35 MPa, about 40 MPa, about 45 MPa, about 50 MPa, about 55 MPa, about 60 MPa, about 65 MPa, about 70 MPa, about 75 MPa, about 80 MPa, about 85 MPa, about 90 MPa, about 95 MPa, about 100 MPa, about 150 MPa, about 200 MPa, about 250 MPa, about 300 MPa, about 350 MPa, about 400 MPa, about 450 MPa, about 500 MPa, about 550 MPa, about 600 MPa, about 650 MPa, about 700 MPa, about 750 MPa, about 800 MPa, about 850 MPa, about 900 MPa, about 950 MPa, about 1000 MPa, or ranges including and/or in between any two of the preceding values.

### Tissue-Engineered Construct

The spine disc replacement of the present technology includes a tissue-engineered construct that includes a bio ink. In any embodiment herein, the tissue-engineered construct may be configured to have any shape or size. For example, in any embodiment herein, the tissue-engineered construct may have a circular shape, ovular shape, elliptical shape, or polygonal shape. In any embodiment herein, the tissue-engineered construct may have a shape of an intervertebral disc (IVD). For example, the IVD shaped tissue-engineered construct may be a single uniform tissue-engineered construct. In any embodiment herein, the IVD shaped tissue-engineered construct may include an annulus fibrosus structure and a nucleus pulposus structure, where the annulus fibrosus structure is circumferentially aligned around the nucleus pulposus. In any embodiment herein, the IVD shaped tissue-engineered construct may include an annulus fibrosus structure, a nucleus pulposus structure, and an endplate structure, where the annulus fibrosus structure is circumferentially aligned around the nucleus pulposus. In any embodiment, the tissue-engineered construct of the present technology may have the shape of an IVD of a subject. In any embodiment, the tissue-engineered construct may have the shape of a negative space between vertebrae of a subject (e.g., between vertebrae as seen in a MRI or CT scan).

The tissue-engineered construct may be configured such that the size of the tissue-engineered construct is greater than the size of the biocompatible support structure, where the tissue-engineered construct is partially or fully in contact with the biocompatible support structure. For example, in any embodiment herein, the tissue-engineered construct may be configured such that the biocompatible support structure is fully or partially encapsulated within the tissue-engineered construct.

In any embodiment herein, the tissue-engineered construct may be configured such that size of the tissue-engineered construct is smaller than the biocompatible support, where the tissue-engineered construct is enclosed by and not in contact with the biocompatible support structure. For example, in any embodiment herein, the tissue-engineered construct may be enclosed by the annular ring of the biocompatible support structure such that the tissue-engineered construct does not come in contact with the inner surface wall of the annular ring.

In any embodiment herein, the tissue-engineered construct may be configured to have a size such that the tissue-engineered construct is partially or fully enclosed by and in contact with one or more surfaces of the biocompatible support structure. For example, in any embodiment herein, the tissue-engineered construct may be configured to have a size such that the tissue-engineered construct contacts the inner surface wall of the annular ring, a planar surface of the first and/or second plate, or a combination thereof. In any embodiment herein, the tissue-engineered construct may have a size that exceeds the capacity of the biocompatible support structure such that the tissue-engineered construct extends (or protrudes) beyond the medial thickness or lateral thickness of the annular ring, or the circumference of the first plate and/or second plate. For example, in any embodiment herein, the tissue-engineered construct may extend (or protrude) through the one or more apertures of the annular ring, first plate, and/or second plate of the biocompatible support structure. In any embodiment herein, the tissue-engineered construct may extend (or protrude) through the interior cavity of the annular ring beyond the first and/or second planar surface of the annular ring, where the biocompatible support structure may include the first plate and/or second plate which may rest on a surface of the tissue-engineered construct or be encapsulated by the tissue-engineered construct without contacting the annular ring. In any embodiment herein, the tissue-engineered construct may be configured to encapsulate the one or more linkages of the biocompatible support structure.

Without being bound by theory, it is believed that when the tissue-engineered construct is configured such that it is enclosed by and in contact with the biocompatible support such that tissue-engineered construct extends (or protrudes) through the one or more apertures of the one or more of the annular ring, the first plate, or the second plate, this creates friction between the biocompatible support structure and the tissue-engineered construct. In this regard, the friction generated prevents slippage of the tissue-engineered construct such that it remains within the biocompatible support. In addition, it is further believed that the protruding portions of the tissue-engineered construct generate friction between the spine disc replacement and the vertebrae end plates, which may also prevent slippage of the spine disc replacement.

In any embodiment herein, the tissue-engineered construct may include a unique set of mechanical properties to enable its proper function. For example, in any embodiment herein, the tissue-engineered construct may have one or more of an equilibrium modulus of about 2 MPa to about 15 MPa, an instantaneous modulus of about 5 kPa to about 2000 kPa, and a hydraulic permeability of about 1×10⁻¹⁶ m²/Pa·s to about 1×10⁻⁸ m²/Pa·s. In any embodiment herein, the tissue-engineered construct may have an equilibrium modulus of about 2 MPa to about 15 MPa, about 2 MPa to about 12 MPa, about 2 MPa to about 10 MPa, about 2 MPa to about 8 MPa, about 2 MPa to about 6 MPa, about 2 MPa to about 4 MPa, or any range including and/or in between any two of the preceding values. For example, in any embodiment herein, the equilibrium modulus may be 2 MPa, about 2.5 MPa, about 3 MPa, about 3.5 MPa, about 4 MPa, about 4.5 MPa, about 5 MPa, about 5.5 MPa, about 6 MPa, about 6.5 MPa, about 7 MPa, about 7.5 MPa, about 8 MPa, about 8.5 MPa, about 9 MPa, about 9.5 MPa, about 10 MPa, about 11 MPa, about 12 MPa, about 13 MPa, about 14 MPa, about 15 MPa, or any range including and/or in between any two of the preceding values.

In any embodiment herein, the tissue-engineered construct may have an instantaneous modulus of about 5 kPa to about 2000 kPa, 5 kPa to about 1500 kPa, about 5 kPa to about 1000 kPa, about 5 kPa to about 500 kPa, about 5 kPa to about 100 kPa, about 5 kPa to about 40 kPa, or any range including and/or in between any two of the preceding values. For example, in any embodiment herein, the tissue engineered construct may have an instantaneous modulus of about 5 kPa, about 6 kPa, about 7 kPa, about 8 kPa, about 9 kPa, about 10 kPa about 12 kPa, about 14 kPa, about 16 kPa, about 18 kPa, about 20 kPa, about 22 kPa, about 24 kPa, about kPa, about 26 kPa, about 28 kPa, about 30 kPa, about 32 kPa, about 34 kPa, about 36 kPa, about 38 kPa, about 40 kPa, about 60 kPa, about 80 kPa, about 100 kPa, about 200 kPa, about 300 kPa, about 400 kPa, about 500 kPa, about 600 kPa, about 700 kPa, about 800 kPa, about 900 kPa, about 1000 kPa, about 1100 kPa, about 1200 kPa, about 1300 kPa, about 1400 kPa, about 1500 kPa, about 1600 kPa, about 1700 kPa, about 1800 kPa, about 1900 kPa, about 2000 kPa, or any range including and/or in between any two of the preceding values. In any embodiment herein, the tissue-engineered construct may have a hydraulic permeability of about 1×10⁻¹⁶ m²/Pa·s to about 1×10⁻⁸ m²/Pa·s, about 1×10⁻¹³ m²/Pa·s to about 9×10⁻¹⁰ m²/Pa·s, about 1×10⁻¹² m²/Pa·s to about 6×10⁻¹⁰, about 1×10⁻¹¹ m²/Pa·s to about 3×10⁻¹⁰ m²/Pa·s, or any range including and/or in between any two of the preceding values.

The tissue-engineered construct may include a bio ink. The term “bio ink” refers to an ink derived from biomaterial. For example, in any embodiment herein, the bio ink may include, but is not limited to, hydrogel (e.g., alginate hydrogel), agarose, collagen, chitosan, fibrin, hyaluronic acid, carrageenan, polyethylene oxide, polypropylene oxide, polyethylene oxide-co-polypropylene oxide, hydroxypropyl methyl cellulose, poly(propylene fumarate-co-ethylene glycol), poly(ethylene glycol)-co-poly(lactic acid), poly(vinyl alcohol), KDLI 2 oligopeptides, poly(n-isopropyl acrylamide), or combinations of two or more thereof. The bio ink may have a controlled rate of crosslinking through the adjustment of environmental variables including, but not limited to, temperature, pH, ionic strength, heat, light, or the addition of chemical crosslinking agents such as calcium, magnesium, barium, chondroitin, sulfate, carbodiimides, ribose, riboflavin, and thrombin. Suitable hydrogels for use in bio inks for tissue-engineered constructs are described in U.S. Pat. No. 9,044,335 entitled “COMPOSITE TISSUE-ENGINEERED INTERVERTEBRAL DISC WITH SELF-ASSEMBLED ANNULAR ALIGNMENT,” filed on May 5, 2010, the entire contents of which are hereby incorporated by reference.

In any embodiment herein, the bio ink of the tissue-engineered construct may include alginate hydrogel. For example, in any embodiment herein, the alginate hydrogel may be present in an amount from about 0.5% (w/v) to about 10% (w/v). For example, in any embodiment herein, the alginate hydrogel may be present in an amount of about 0.5% (w/v), about 0.6% (w/v), about 0.7% (w/v), about 0.8% (w/v), about 0.9% (w/v), about 1.0% (w/v), about 1.5% (w/v), about 2.0% (w/v), about 2.5% (w/v), about 3.0% (w/v), about 3.5% (w/v), about 4.0% (w/v), about 4.5% (w/v), about 5.0% (w/v), about 5.0% (w/v), about 5.5% (w/v), about 6.0% (w/v), about 6.5% (w/v), about 7.0% (w/v), about 7.5% (w/v), about 8.0% (w/v), about 8.5% (w/v), about 9.0% (w/v), about 9.5% (w/v), about 10.0% (w/v), or any range including and/or in between any two the preceding values.

In any embodiment herein, the bio ink of the tissue-engineered construct may include collagen. Collagen-based materials are advantageous for use in tissue-engineered constructs. Methods of harvesting collagen for use in bio gel compositions, methods for making 3D structures using bio gel compositions, and methods for preparing bio gel compositions for use in 3D printing systems are described in PCT Application Serial No. PCT/US2017/034582 entitled “3D PRINTABLE BIO GEL AND METHOD OF USE,” filed on May 25, 2017 and having Attorney Docket No. 113066-0104, the entire contents of which are hereby incorporated by reference for the background information and methods set forth therein.

In any embodiment herein, the bio ink of the tissue-engineered construct may include collagen in an amount greater than about 5 mg/ml. Suitable amounts of collagen in the bio ink may include, but are not limited to, about 5 mg/ml to about 200 mg/ml. For example, in any embodiment herein, the collagen may be present in an amount of about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, about 160 mg/ml, about 165 mg/ml, about 170 mg/ml, about 175 mg/ml, about 180 mg/ml, about 185 mg/ml, about 190 mg/ml, about 195 mg/ml, about 200 mg/ml, or any range including and/or in between any two of the preceding values.

In any embodiment herein, the tissue-engineered construct may include bio ink having type I collagen, type II collagen, or a combination thereof. For example, in any embodiment herein, when the tissue-engineered construct includes an annulus fibrosus structure and a nucleus pulposus structure, the annulus fibrosus structure may include type I collagen and the nucleus pulposus structure may include type II collagen.

In any embodiment herein, the bio ink of the tissue-engineered construct may further include a neutralizer and cellular media as described herein in any embodiment. Suitable neutralizers may include, but are not limited to, formulations containing weak acid, for instance. In any embodiment herein, the tissue-engineered construct may be cellular or acellular. For example, in any embodiment herein, the bio ink of the tissue-engineered construct may include cellular media containing a population of cells which may include, but are not limited to, living cells obtained and/or isolated from intervertebral disc tissue, such as, nucleus pulposus or annulus fibrosus. The cellular media may further include other living cells; for example, in any embodiment herein, the bio ink may include epidermal cells, chondrocytes and other cells that form mesenchymal stem cells, IVD stem cells, cartilage, macrophages, adipocytes, dermal cells, muscle cells, hair follicles, fibroblasts, organ cells, osteoblasts, osteocytes and other cells that form bone, endothelial cells, mucosal cells, pleural cells, ear canal cells, tympanic membrane cells, peritoneal cells, Schwann cells, corneal epithelial cells, gingiva cells, central nervous system neural stem cells, tracheal epithelial cells, or combinations of two or more thereof. In any embodiment herein, the bio ink of the tissue-engineered construct may be acellular, where the bio ink does not contain living cells. In any embodiment herein, the tissue-engineered construct may be configured such that the tissue-engineered construct has areas or components (e.g., annulus fibrosus, nucleus pulposus, or end plate) that may be acellular or cellular.

In any embodiment herein, the population of cells may be present in an amount from about 1.0×10⁵ cells/ml to about 5.0×10⁷ cells/ml. For example, in any embodiment herein, the amount of cells present in the bio ink of the tissue-engineered construct may include about 1.0×10⁵ cells/ml, about 2.0×10⁵ cells/ml, about 3.0×10⁵ cells/ml, about 4.0×10⁵ cells/ml, about 5.0×10⁵ cells/ml, about 6.0×10⁵ cells/ml, about 7.0×10⁵ cells/ml, about 8.0×10⁵ cells/ml, about 9.0×10⁵ cells/ml, about 1.0×10⁶ cells/ml, about 2.0×10⁶ cells/ml, about 3.0×10⁶ cells/ml, about 4.0×10⁶ cells/ml, about 5.0×10⁶ cells/ml, about 6.0×10⁶ cells/ml, about 7.0×10⁶ cells/ml, about 8.0×10⁶ cells/ml, about 9.0×10⁶ cells/ml, about 1.0×10⁷ cells/ml, about 2.0×10⁷ cells/ml, about 3.0×10⁷ cells/ml, about 4.0×10⁷ cells/ml, about 5.0×10⁷ cells/ml, or any range including and/or in between any two of the preceding values. Suitable amounts of cells present in the bio ink of the tissue-engineered construct include, but are not limited to about 1.0×10⁵ cells/ml to about 5.0×10⁷ cells/ml, about 1.0×10⁵ cells/ml to about 1.0×10⁷ cells/ml, about 1.0×10⁵ cells/ml to about 5.0×10⁶ cells/ml, about 1.0×10⁵ cells/ml to about 5.0×10⁶ cells/ml, or any range including and/or in between any two of the preceding values.

In any embodiment herein, the bio ink of the tissue-engineered construct may further include carriers or additives. Suitable carriers may include, but are not limited to, water, aqueous ionic salt solutions (e.g., sodium hydroxide), phosphate buffer saline (PBS), cell medium, fetal bovine serum (FBS), Dulbecco's minimum essential medium (DMEM), fibroblast growth factor (bFGF), or the like or combinations thereof. Suitable additives include, but are not limited to, growth factors and crosslinking agents. Suitable crosslinking agents include, but are not limited to, riboflavin, ribose, polyethylene glycol (PEG), glutaraldehyde, 1-ethyl-3-(3-dimethylaminopropyl)-carbodiimide hydrochloride, genipin, chitosan, or the like or combinations thereof.

The spine disc replacement composition includes the tissue-engineered construct and biocompatible support structure together, in any configuration as disclosed herein, and possesses improved mechanical properties (i.e., strength). The spine disc replacement, in any embodiment herein, may withstand an axial compression of about 1 kN to about 10,000 kN and a shear force of about 0.2 kN to about 1,000 kN. For example, in any embodiment herein, the spine disc replacement of the present technology may withstand an axial compression of about 1 kN, 2 kN, about 3, kN, about 4 kN, about 5 kN, about 6 kN, about 7 kN, about 8 kN, about 9 kN, about 10 kN, about 20 kN, about 30 kN, about 40 kN, about 50 kN, about 60 kN, about 70 kN, about 80 kN, about 90 kN, about 100 kN, about 200 kN, about 300 kN, about 400 kN, about 500 kN, about 600 kN, about 700 kN, about 800 kN, about 900 kN, about 1000 kN, about 1500 kN, about 2000 kN, about 2500 kN, about 3000 kN, about 3500 kN, about 4000 kN, about 4500 kN, about 5000 kN, about 5500 kN, about 6000 kN, about 6500 kN, about 7000 kN, about 7500 kN, about 8000 kN, about 8500 kN, about 9000 kN, about 9500 kN, about 10, 000 kN, or ranges including and/or in between any two of the preceding values. Suitable axial compression ranges may include but are not limited to about 1 kN to about 10,000 kN, about 1 kN to about 5000 kN, about 1 kN to about 1000 kN, about 1 kN to about 500 kN, about 1 kN to about 15 kN, about 4 kN to about 10 kN, or ranges including and/or in between any two of the preceding values.

In any embodiment herein, the spine disc replacement of the present technology may withstand a shear force of about 0.2 kN to about 1,000 kN. For example, in any embodiment herein, the spine disc replacement of the present technology may withstand a shear force may be about 0.2 kN, about 0.3 kN, about 0.4 kN, about 0.5 kN, about 0.6 kN, about 0.7 kN, about 0.8 kN, about 0.9 kN, about 1.0 kN, about 10 kN, about 50 kN, about 100 kN, about 150 kN, about 200 kN, about 250 kN, about 300 kN, about 350 kN, about 400 kN, about 450 kN, about 500 kN, about 550 kN, about 600 kN, about 650 kN, about 700 kN, about 750 kN, about 800 kN, about 850 kN, about 900 kN, about 950 kN, about 1000 kN, or ranges including and/or in between any two of the preceding values. Suitable ranges of the shear force include, but are not limited to, about 0.2 kN to about 1000 kN, about 1 kN to about 500 kN, about 10 kN to about 100 kN, about 0.2 kN to about 0.9 kN, or ranges including and/or in between any two of the preceding values.

### Methods

In a related aspect, the present technology provides a method of fabricating the biocompatible support structure as described herein in any embodiment, the method includes depositing a biocompatible material to a substrate; optionally crosslinking the deposited biocompatible material; and optionally repeating the depositing and optional crosslinking steps to obtain the biocompatible support structure; where the biocompatible support structure includes one or more of an annular ring, a first plate, or a second plate of a biocompatible material, the annular ring includes an inner surface wall, an outer surface wall, a first planar surface, and a second planar surface, and the biocompatible material is present in an amount of about 1% to about 100% by weight of the biocompatible support structure.

The method of fabricating the biocompatible support structure may include one or more fabrication systems or methods, including but not limited to, injection molding, rotational molding, positive molds, negative molds, extrusion, subtractive manufacturing, milling and machining, and three-dimensional (3D) printing. For example, in any embodiment herein, the method of fabricating may include 3D printing. Suitable 3D printing methods may include, but is not limited to, ink-jet printing, layer-by-layer printing, extrusion printing, or bioprinting. For example, in any embodiment herein, the depositing may include depositing one or more layers of biocompatible material. For example, in any embodiment herein, 3D printing may include: depositing one or more layers of a biocompatible material to a substrate; optionally crosslinking the deposited biocompatible material; and optionally repeating the depositing and optional crosslinking steps to obtain the biocompatible support structure; where the biocompatible support structure includes one or more of an annular ring, a first plate, or a second plate of a biocompatible material, the annular ring includes an inner surface wall, an outer surface wall, a first planar surface, and a second planar surface, and the biocompatible material is present in an amount of about 1% to about 100% by weight of the biocompatible support structure.

In any embodiment herein, the biocompatible support structure may be fabricated using biocompatible materials as described herein in any embodiment using 3D printing systems as described in PCT Application Serial No. PCT/2018/034457 entitled “ASEPTIC PRINTER SYSTEM INCLUDING DUAL ARM-MECHANISM,” filed on May 24, 2018 and having Attorney Docket No. 113066-0107, the entire contents of which are hereby incorporated by reference for the background information and methods set forth therein.

In any embodiment herein, the method of fabricating the biocompatible support structure may include injection molding. For example, in any embodiment herein, the depositing the biocompatible material may include injection molding. In any embodiment herein, the depositing may include depositing the biocompatible material in a mold of the biocompatible support structure; optionally crosslinking the biocompatible material in the mold, and optionally repeating the depositing and optional crosslinking steps; and removing the biocompatible support structure from the mold.

In any embodiment herein, the substrate may include a surface of a 3D printer (e.g., build plate of a 3D printer). In any embodiment herein, the substrate may include a mold suitable for use in one or more of injection molding, rotational molding, positive molds, negative molds, or the like. In any embodiment herein, the substrate may include a surface of a fabrication device suitable for performing subtractive manufacturing, milling and machining, or the like.

In any embodiment herein, the biocompatible material is crosslinked. For example, in any embodiment herein, the biocompatible material may be subjected to crosslinking conditions to produce a cross-linked or polymerized biocompatible material. Suitable crosslinking conditions may include, but are not limited to, UV exposure. In any embodiment herein, the biocompatible material may not include crosslinking. For example, in any embodiment, following deposition, the biomaterial may include a cooling step, a fusing step, and the like or combinations thereof.

In the method of the present technology, the method of fabricating the biocompatible support structure may include depositing the one or more layers of biocompatible material such that the annular ring has a medial thickness of about 100 μm to about 6000 μm and a lateral thickness of about 100 μm to about 1000 μm as described herein in any embodiment, and the first plate and the second plate have a thickness from about 100 μm to about 600 μm as described herein in any embodiment.

In any embodiment herein, the method of fabricating the biocompatible support structure may include depositing the one or more layers of biocompatible material such that the biocompatible support structure has the circumferential shape of an IVD. For example, in any embodiment, the biocompatible support structure may have the circumferential shape of an IVD of a subject. Typically, the subject or patient is a human, and preferably in need of implants for IVD tissue replacement and/or regeneration.

In the method of the present technology, the biocompatible material may include a biocompatible material as described herein in any embodiment. For example, in any embodiment herein, the biocompatible material may include, but is not limited to, polysaccharides, biocompatible polymers, rubber, silicon, biocompatible metals (e.g., steel, cobalt-chromium alloys, titanium, titanium alloys magnesium, magnesium alloys, zinc, zinc alloys, iron alloys, or the like or combinations thereof), biocompatible ceramics, polyethylene glycol, polypropylene glycol, poly amino acids, natural and biopolymers (e.g., glycosaminoglycans, cellulose, chitosan, chitin, dextrans, gelatin, collagen, lignins, polyamino acids, glycoproteins, eslastin, laminins), or combinations of two or more thereof. In any embodiment, the biocompatible material may include biocompatible polymers; for example, in any embodiment herein, the biocompatible polymers may include polylactides (PLA), polyglycolides acid (PGA), poly (lactide-co-glycolide) (PLGA), polydioxanone (PDO), polycaprolactones, and combinations thereof). In any embodiment herein, the biocompatible polymer may have a melting point between about 50° C. to about 290° C. For example, in any embodiment herein, the biocompatible polymer may have a melting point of about 50° C., about 55° C., about 60° C., about 65° C., about 70° C., about 75° C., about 80° C., about 85° C., about 90° C., about 95° C., about 100° C., about 110° C., about 120° C., about 130° C., about 140° C., about 150° C., about 160° C., about 170° C., about 180° C., about 190° C., about 200° C., about 210° C., about 220° C., about 230° C., about 240° C., about 250° C., about 260° C., about 270° C., about 280° C., about 290° C., or any range including and/or in between any two of the preceding values.

In any embodiment herein, the biocompatible support structure obtained from the method of fabricating may have one or more of a flexural modulus of about 0.2 GPa to about 100 GPa, a modulus of elasticity of about 0.2 GPa to about 100 GPa, or a tensile strength of about 1 MPa to about 1000 MPa as described herein in any embodiment.

In any embodiment herein, the method may further include sterilizing the biocompatible support structure. For example, in any embodiment herein, the sterilizing may include, but is not limited to, gamma irradiation, incubation with peracid, autoclaving, UV irradiation, peroxide sterilization, supercritical fluid treatment, and the like or combinations thereof.

In another related aspect, the present technology provides a method of fabricating the spine disc replacement that includes fabricating a biocompatible support structure as described herein in any embodiment comprising: depositing a biocompatible material to a substrate; optionally crosslinking the deposited biocompatible material; and optionally repeating the depositing and optional crosslinking steps to obtain the biocompatible support structure; fabricating a tissue-engineered construct as described herein in any embodiment comprising: depositing a bio ink as described herein in any embodiment in or around the biocompatible support structure; crosslinking the bio ink, and optionally repeating the depositing and crosslinking steps, to form the tissue-engineered construct; and curing the spine disc replacement composition; where the biocompatible support structure includes one or more of an annular ring, a first plate, or a second plate of a biocompatible material, the annular ring includes an inner surface wall, an outer surface wall, a first planar surface, and a second planar surface, and the biocompatible material is present in an amount of about 1% to about 100% by weight of the biocompatible support structure.

The method of fabricating the biocompatible support structure may include the methods described herein in any embodiment. For example, in any embodiment herein, the one or more fabrication systems or methods may include, but are not limited to, injection molding, rotational molding, positive molds, negative molds, subtractive manufacturing, milling and machining, and three-dimensional (3D) printing. In any embodiment herein, the method of fabricating may include 3D printing as described herein in any embodiment. In any embodiment herein, the method of fabricating the biocompatible support structure may include injection molding as described herein in any embodiment. In any embodiment herein, the method may include crosslinking the biocompatible material as described herein in any embodiment.

The fabricating of the tissue-engineered construct includes depositing one or more layers of a bio ink as described herein in any embodiment. In any embodiment herein, the fabricating the tissue-engineered construct may include one or more fabrication systems or methods; for example, in any embodiment herein, the one or more fabrication systems or methods may include injection molding, rotational molding, positive molds, negative molds, subtractive manufacturing, milling and machining, and 3D printing. In any embodiment herein, the method of fabricating may include 3D printing as described herein in any embodiment. For example, in any embodiment herein, the depositing may include depositing one or more layers of bio ink in or around the biocompatible support structure using a 3D printer; crosslinking the bio ink, and optionally repeating the depositing and crosslinking steps, to obtain the tissue-engineered construct; and curing the spine disc replacement.

In any embodiment herein, fabricating the tissue-engineered construct may include injection molding. For example, in any embodiment herein, the depositing may include depositing the bio ink in a mold containing the biocompatible support structure; crosslinking the bio ink in the mold, and optionally repeating the depositing and crosslinking steps, to obtain the tissue-engineered construct; and curing the spine disc replacement, where the mold may be removed prior to or after the curing step. In any embodiment herein, the method may include removing the mold after curing the spine disc replacement. In any embodiment herein, the method may include removing the mold before curing the spine disc replacement.

The method may include fabricating a tissue-engineered construct having an annulus fibrosus structure and a nucleus pulposus structure as described herein in any embodiment. In any embodiment, the method of fabricating may include depositing the bio ink to form the annulus fibrosus structure and depositing the bio ink to form the nucleus pulposus structure. For example, in any embodiment herein, the method may include depositing the bio ink to form the annulus fibrosus structure and nucleus pulposus structure concurrently or sequentially. In some embodiments, the depositing may include forming the annulus fibrosus structure and nucleus pulposus structure sequentially. In any embodiment herein, the depositing may include forming the annulus fibrosus structure and nucleus pulposus structure concurrently.

In any embodiment herein, the bio ink is crosslinked. For example, in any embodiment herein, the bio ink may be subjected to crosslinking conditions to produce a crosslinked or polymerized bio ink. For example, in any embodiment herein, the crosslinking may include UV irradiation, addition of salts, neutralization of pH, thermal crosslinking, and the like or combinations thereof.

The spine disc replacement undergoes a curing step following crosslinking. In any embodiment herein, the method includes curing the spine disc replacement at a temperature of about 34° C. to about 37° C. Suitable curing temperatures may include, but are not limited to, about 34° C. to about 37° C., about 35° C. to about 37° C., about 36° C. to about 37° C., and any range including and/or in between any two of the preceding values. In any embodiment herein, the spine disc replacement may be placed in a buffer solution or cell medium during the curing step. In any embodiment herein, suitable buffer solutions include, but are not limited to, phosphate buffered saline, sodium chloride solution, phosphates, and phosphate buffer solution. Suitable cell medium may include, but is not limited to, serum, serum-free medium, HEPES, DMEM, bFGF, FBS, and the like or combinations thereof. In any embodiment herein, the method may include curing the spine disc replacement in buffer solution and cell medium. In any embodiment herein, the curing may occur over a period of about 3 h to about 24 h. For example, in any embodiment herein, the curing period may be from about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h, or any range including and/or in between any two of the preceding values.

In any embodiment herein, bio ink may include, but is not limited to, alginate, agarose, collagen, chitosan, fibrin, hyaluronic acid, carrageenan, polyethylene oxide, polypropylene oxide, polyethylene oxide-co-polypropylene oxide, hydroxypropyl methyl cellulose, polypropylene fumarate-co-ethylene glycol), poly(ethylene glycol)-co-poly(lactic acid), poly(vinyl alcohol), KDLI 2 oligopeptides, poly(n-isopropyl acrylamide), or combinations of two or more thereof. The bio ink may have a controlled rate of crosslinking through the adjustment of environmental variables including, but not limited to, temperature, pH, ionic strength, heat, light, or the addition of chemical crosslinking agents such as calcium, magnesium, barium, chondroitin, sulfate, and thrombin. Suitable hydrogels for use in bio inks for tissue-engineered constructs are described in U.S. Pat. No. 9,044,335 entitled “COMPOSITE TISSUE-ENGINEERED INTERVERTEBRAL DISC WITH SELF-ASSEMBLED ANNULAR ALIGNMENT,” filed on May 5, 2010, the entire contents of which are hereby incorporated by reference.

In any embodiment herein, the bio ink may include alginate hydrogel as described herein in any embodiment. For example, in any embodiment herein, the alginate hydrogel may be present in an amount from about 0.5% (w/v) to about 10% (w/v). In any embodiment herein, the bio ink may include collagen as described herein in any embodiment. For example, in any embodiment herein, the bio ink may include collagen in an amount greater than about 5 mg/ml. In any embodiment herein, the bio ink may include type I collagen, type II collagen, or a combination thereof. For example, in any embodiment herein, the method of fabricating the tissue-engineered construct may include depositing one or more layers of bio ink that includes type I collagen to form an annulus fibrosus structure and depositing one or more layers of a bio ink that includes type II collagen to form a nucleus pulposus structure.

In any embodiment herein, the bio ink may further include a neutralizer and cellular media as described herein in any embodiment. Suitable neutralizers may include, but are not limited to, formulations containing weak acid, for instance. In any embodiment herein, the bio ink may be cellular or acellular. For example, in any embodiment herein, the bio ink may include cellular media containing living cells which may include, but are not limited to, cells obtained and/or isolated from intervertebral disc tissue, such as, nucleus pulposus or annulus fibrosus. The cellular media may further include other living cells; for example, in any embodiment herein, the bio ink may include epidermal cells, chondrocytes and other cells that form mesenchymal stem cells, IVD stem cells, cartilage, macrophages, adipocytes, dermal cells, muscle cells, hair follicles, fibroblasts, organ cells, osteoblasts, osteocytes and other cells that form bone, endothelial cells, mucosal cells, pleural cells, ear canal cells, tympanic membrane cells, peritoneal cells, Schwann cells, corneal epithelial cells, gingiva cells, central nervous system neural stem cells, tracheal epithelial cells, or combinations of two or more thereof. In any embodiment herein, the bio ink may be acellular, where the bio ink does not contain living cells.

In any embodiment herein, the bio ink may further include carriers or additives. Suitable carriers may include, but are not limited to, water, aqueous ionic salt solutions (e.g., sodium hydroxide), phosphate buffer saline (PBS), cell medium, fetal bovine serum (FBS), Dulbecco's minimum essential medium (DMEM), fibroblast growth factor (bFGF), or the like or combinations thereof. Suitable additives include, but are not limited to, crosslinking agents. Suitable crosslinking agents include, but are not limited to, riboflavin, ribose, polyethylene glycol (PEG), glutaraldehyde, 1-ethyl-3-(3-dimethylaminopropyl)-carbodiimide hydrochloride, genipin, chitosan, or the like or combinations thereof.

In any embodiment herein, the bio ink is crosslinked. For example, in any embodiment herein, the bio ink may be subjected to crosslinking conditions to produce a crosslinked or polymerized bio ink material.

In any embodiment herein, the method of fabricating the biocompatible support structure may include depositing the one or more layers of biocompatible material such that the biocompatible support structure has the circumferential shape of an IVD. For example, in any embodiment, the biocompatible support structure may have the circumferential shape of an IVD of a subject. Typically, the subject or patient is a human, and preferably in need of implants for IVD tissue replacement and/or regeneration.

In any embodiment herein, the spine disc replacement may withstand one or more of an axial compression of about 1 kN to about 10,000 kN or a shear force of about 0.2 kN to about 1,000 kN as described herein in any embodiment.

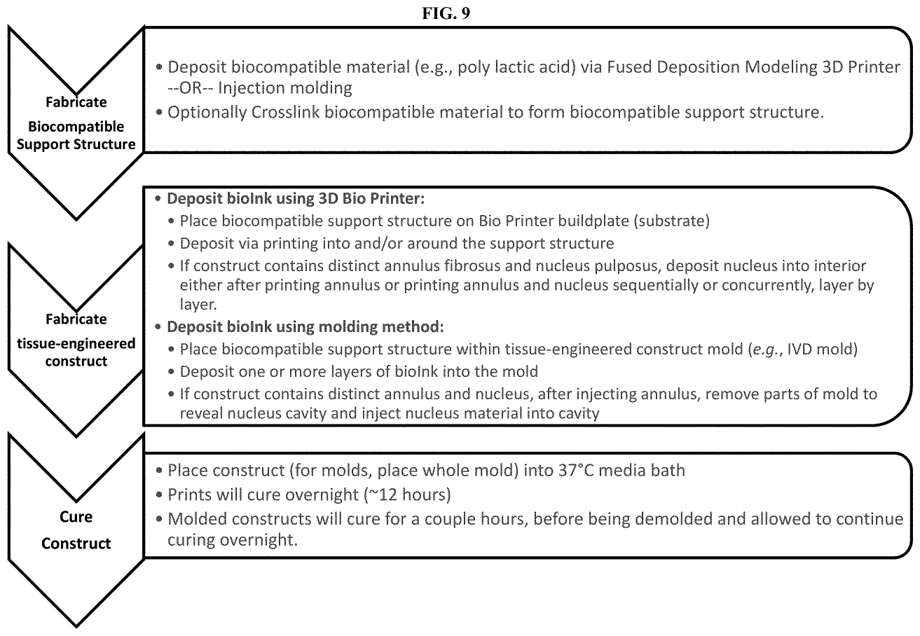

In at least one embodiment, the methods of fabricating a spine disc replacement of the present technology is a method according to the steps illustrated in FIG. 9.

The present invention, thus generally described, will be understood more readily by reference to the following examples, which are provided by way of illustration and are not intended to be limiting of the present invention.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

While this specification contains many specific implementation details, these should not be construed as limitations on the scope of what may be claimed, but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The terms “coupled,” “connected,” and the like as used herein mean the joining of two components directly or indirectly to one another. Such joining may be stationary (e.g., permanent) or moveable (e.g., removable or releasable). Such joining may be achieved with the two components or the two components and any additional intermediate components being integrally formed as a single unitary body with one another or with the two components or the two components and any additional intermediate components being attached to one another.

The terms “fluidly coupled,” “in fluid communication,” and the like as used herein mean the two components or objects have a pathway formed between the two components or objects in which a fluid, such as water, air, etc., may flow, either with or without intervening components or objects. Examples of fluid couplings or configurations for enabling fluid communication may include piping, channels, or any other suitable components for enabling the flow of a fluid from one component or object to another.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A biocompatible support structure for spine disc replacement comprising:
one or more of an annular ring, a first plate, or a second plate of a biocompatible material, wherein:
the annular ring comprises an inner surface wall, an outer surface wall, a first planar surface, and a second planar surface; and
the biocompatible material is present in an amount of about 1% to about 100% by weight of the biocompatible support structure.

2. The support structure of claim 1, wherein the annular ring has a medial thickness of about 100 μm to about 6000 μm and a lateral thickness of about 100 μm to about 2000 μm.

3. The support structure of claim 1, wherein the first plate and the second plate have a thickness from about 100 μm to about 1200 μm.

4. The support structure of claim 1, wherein the support structure has one or more of:
a flexural modulus of about 0.2 GPa to about 100 GPa;
a modulus of elasticity of about 0.02 GPa to about 100 GPa; and/or
a tensile strength of about 1 MPa to about 1000 MPa.

5. The support structure of any claim 1, wherein the biocompatible material is selected from the group consisting of polysaccharides, biocompatible polymers, rubber, silicon, biocompatible metals, biocompatible ceramics, polyethylene glycol, polypropylene glycol, polyamino acids, natural and biopolymers, or combinations of two or more thereof.

6. The support structure of claim 5, wherein:
the biocompatible material is selected from the group consisting of PLA, PGA, PLGA, PDO, polycaprolactones, bioresorbable metal alloys, and combinations thereof; and
the biocompatible material degrades, absorbs, or resorbs at a rate of about 1 month to about 7 years.

7. The support structure of claim 1, wherein the annular ring is configured to have the shape of an intervertebral disc of a subject.

8. The support structure of claim 1, wherein the annular ring has one or more apertures having an average size of about 10 μm to about 10,000 μm.

9. The support structure of claim 1, wherein the support structure further comprises:
one or more appendage elements connected to and extending distally from the first planar surface or the second planar surface of the annular ring or a planar surface of the first plate or the second plate; and/or
one or more linking elements connecting a cross-section of the annular ring from one area of the inner surface wall to another area of the inner surface wall, or connecting a planar surface of the first plate to a planar surface of the second plate.

10. A method for fabricating a biocompatible support structure for spine disc replacement according to claim 1, the method of fabricating comprising:
depositing one or more layers of a biocompatible material to a substrate;
crosslinking the biocompatible material; and
optionally repeating the depositing and crosslinking steps to obtain the biocompatible support structure.

11. The method of claim 10, wherein the method of fabricating comprises one or more of injection molding, rotational molding, molding using positive molds, molding using negative molds, subtractive manufacturing, milling and machining, and three-dimensional (3D) printing.

12. The method of claim 11, wherein:
the depositing comprises 3D printing the one or more layers of biocompatible material; and
the 3D printing is selected from the group consisting of ink-jet printing, layer-by-layer printing, extrusion printing, and bioprinting.

13. The method of claim 11, wherein the method of fabricating comprises injection molding, wherein the injection molding comprises:
depositing one or more layers of the biocompatible material to a substrate, wherein the substrate is a mold of the biocompatible support structure;
crosslinking the biocompatible material in the mold, and optionally repeating the depositing and crosslinking steps; and
removing the biocompatible support structure from the mold.

14. A spine disc replacement composition comprising:
a biocompatible support structure for spine disc replacement according to claim 1; and
a tissue-engineered construct comprising a bio ink.

15. The spine disc replacement composition of claim 14, wherein the bio ink comprises one or more of a hydrogel, agarose, collagen, chitosan, fibrin, hyaluronic acid, carrageenan, polyethylene oxide, polypropylene oxide, polyethylene oxide-co-polypropylene oxide, hydroxypropyl methyl cellulose, poly(propylene fumarate-co-ethylene glycol), poly(ethylene glycol)-co-poly(lactic acid), poly(vinyl alcohol), KDLI 2 oligopeptides, poly(n-isopropyl acrylamide), or combinations of two or more thereof.

16. The spine disc replacement composition of claim 15, wherein the bio ink comprises:
an alginate hydrogel present in an amount of about 0.5% (w/v) to about 10% (w/v); and/or
about 5 mg/ml to about 200 mg/ml of collagen.

17. The spine disc replacement composition of claim 14, wherein the tissue-engineered construct comprises a nucleus pulposus structure comprising type II collagen, and an annulus fibrosus structure comprising type I collagen.

18. The spine disc replacement composition of claim 14, wherein the tissue-engineered construct further comprises a population of cells present in a concentration of about 1.0×10⁵ cells/ml to about 5.0×10⁷ cells/ml.

19. The spine disc replacement composition of claim 14, wherein:
the annular ring of the biocompatible support structure increases the axial stiffness of the tissue-engineered construct by about 5 times to about 10,000 times stiffness; and/or
the first plate or second plate of the biocompatible support structure increases the axial load capacity of the tissue-engineered construct by about 1% to about 10,000%; and/or
the composition withstands an axial compression of about 1 kN to about 10,000 kN, a shear force of about 0.2 kN to about 1,000 kN, or a combination thereof.

20. A method of fabricating the spine disc replacement composition according to claim 14, comprising:
fabricating the biocompatible support structure, comprising:
depositing the biocompatible material to a substrate;
optionally crosslinking the biocompatible material; and
optionally repeating the depositing and optional crosslinking steps to obtain the biocompatible support structure;
fabricating the tissue-engineered construct comprising:
depositing a bio ink in or around the biocompatible support structure;
crosslinking the bio ink, and optionally repeating the depositing and crosslinking steps, to form the tissue-engineered construct; and
curing the spine disc replacement composition.
